(19) United States
(12) Patent Application Publication
(10) Pub. No.: US 2009/0275796 A1
(43) Pub. Date: Nov. 5, 2009

(54) STIMULATION DEVICE

(76) Inventor: Noam Gil, Moshav Givaton (IL)

Correspondence Address: Pearl Cohen Zedek Latzer, LLP, 1500 Broadway, 12th Floor, New York, NY, 10036, US

(21) Appl. No.: 12/435,467

(22) Filed: May 5, 2009

Related U.S. Application Data
(63) Continuation-in-part of application No. PCT/IL2009/000028, filed on Jan. 8, 2009.
Application No. PCT/IL2009/000028 is a continuation-in-part of application No. 11/671,005, filed on Feb. 5, 2007, now Pat. No. 7,534,203.
(60) Provisional application No. 61/019,625, filed on Jan. 8, 2008.

(51) Int. Cl. A61F 5/00

(52) U.S. Cl. 600/38

(57) ABSTRACT

The present invention provides a device having a body, at least one accessory, at least one pump, and at least one tube located within the body and operatively connecting the accessory to the pump. The pump, inflates the accessory thus alters at least one aspect of the accessory. Additionally, the invention provides a device which offers similar features as the previous device but in a sheath-like structure that fits over a phallus or a phallus like structure.

### FIELD OF THE INVENTION

This invention relates generally to stimulation devices and specifically to such devices that incorporate accessories whose shapes, sizes, and other features may be altered.

### BACKGROUND OF THE INVENTION

Most known stimulation devices that comprise movable accessories are operated by an electrical power source. A sensual vibrator is an electrically driven, mechanical device designed to generate vibrations for enhancing sexual pleasure. The vibration is often generated by an electric motor with an unbalanced weight on its driveshaft, wherein the rotating weight causes vibrations when the motor is switched on.

Such vibrators are frequently battery operated. A commonly found design for 15 commercially available vibrators is a cylindrical body with rounded distal end.

To further enhance the effects of the vibrator, a user may opt to employ one or more accessories in conjunction with that vibrator. The terms “accessory” and “accessories” refer herein to one or more objects that are affixed to or otherwise added to the body of a vibrator in order to enhance the sensation produced by that vibrator.

However, most vibrators currently available on the market do not normally include accessories. For those vibrators that do include accessories, the accessories usually are permanently attached to the vibrator, which eliminates the possibility of making adjustments to the size or otherwise altering the accessories. Even when the accessories may be exchanged, adjusted or otherwise altered, that alteration requires a temporary break in the use of the vibrator while the change is effected. This break in continuity may be undesirable for the user and may affect the overall efficacy to the vibrator.

### SUMMARY OF THE INVENTION

The present invention provides a device having a male sex organ shape for providing sexual stimulation in a subject, comprising: an inflatable accessory or a pressure driven accessory; a pump comprising an inflator operably coupled to a tube; a tube having a proximal end and a distal end, wherein the distal end of the tube is operably coupled to the pump, and the proximal end of the tube is operably linked to the accessory, wherein upon actuating the pump the volume of the accessory, is increased.

In another embodiment, the present invention further provides a device comprising: a body; at least one accessory; at least one pump; and at least one tube located within the body and operatively connecting said accessory to the pump; wherein the accessory is imbedded in the body and the activation of at least one pump alters at least one aspect of the accessory.

In another embodiment, the present invention further provides a device comprising: a sheath; at least one accessory; at least one pump; and at least one tube located within the sheath and operatively connecting said accessory to the pump; wherein the accessory is imbedded in the sheath and the activation of at least one pump alters at least one aspect of the accessory.

### BRIEF DESCRIPTION OF THE DRAWINGS

FIG. 1A is a perspective view of an embodiment of the device (10) of the present invention, wherein the stimulating accessories are not inflated. Pump (20) comprises a testicle shape; FIG. 1B is a perspective view of an embodiment, wherein the stimulating accessories are inflated showing device (10) comprising a body 12, at least one accessory (30) imbedded in body (12) and at least one pump (20).

FIG. 2A is a cross-sectional, perspective view of an embodiment of the present invention, equipped with a single tube operatively connecting a plurality of accessories to a single pump; Device 10 comprises a body (12), at least one accessory (30) imbedded in body (12) and at least one pump 20. Tube (22) located within body (12) interconnects pump (20) to at least one accessory (30). FIG. 2B is a cross-sectional, perspective view of the embodiment of FIG. 2A, wherein the accessories are inflated by the pump.

FIGS. 3A and 3B are perspective inner views of the embodiment of FIG. 2B, with the cover partially cut away in order to better describe the air pumping mechanism. Selector switch (70) enables flow from pump (20) to tube (22).

FIGS. 4A and 4B are perspective views of another embodiment of the present invention, equipped the inflating/deflating mechanism including the pump (20), a valve (80), a valve housing and a selector switch (70), and inner one way air stopper.

FIG. 5 is another embodiment of the present invention, equipped with a tube operatively connecting inflatable spiral shaped accessory (30). Upon inflating, spiral shaped accessory (30) extends from body (12) outwards and extends the length of device (10) by pressure lifting cup (40).

FIG. 6 is another embodiment of the present invention, equipped with an electrical pump (20) controlled by selector switch (70). Tube (22) splits to pressure accessory (30). Actuating the device causes accessory (30) to extend outwards beyond body (12).

The drawings together with the description make apparent to those skilled in the art how the invention may be embodied in practice. No attempt is made to show structural details of the invention in more detail than is necessary for a fundamental understanding of the invention. It will be appreciated that for simplicity and clarity of illustration, elements shown in the figures have not necessarily been drawn to scale. For example, the 5 dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In one embodiment, the invention provides a sexual stimulation device comprising a body; at least one accessory; at least one accessory pump; and at least one tube located within the body and operatively connecting at least one accessory to at least one pump. In another embodiment, at least one accessory is imbedded within the body of the device. In another embodiment, at least one accessory is partially imbedded within the body of the device. In another embodiment, the activation of at least one pump alters at least one aspect of at least one accessory. In another embodiment, the activation of at least one pump inflates at least one accessory. In another embodiment, the activation of at least one pump increases a volume of at least one accessory. In another embodiment, the accessory projects from the device once activated.

In another embodiment, activating an accessory comprises inflating the accessory. In another embodiment, the accessory once activated increases the diameter of the device. In another embodiment, the accessory once activated increases the diameter of the body of the device. In another embodiment, the accessory once activated increases the length of the device. In another embodiment, the accessory once activated increases the diameter of the body of the device. In another embodiment, the accessory once activated increases the diameter and length of the body of the device. In another embodiment, the accessory once activated increases the diameter and length of the device.

In another embodiment, the device comprises a rigid penis shaped material and a less rigid inflatable material that is inserted within the rigid penis shaped material. In another embodiment, a rigid penis shaped material comprises openings. In another embodiment, the less rigid inflatable material protrudes outwards via an opening in the rigid penis shaped material thus forming an inflatable accessory. In another embodiment, the device comprising a penis shape material comprises areas that become more rigid upon inflation. In another embodiment, the device comprising a rigid penis shaped material comprises opening having different shapes. In another embodiment, the material being inflated comprises various colors. In another embodiment, the material being inflated comprises various textures. In another embodiment, the characteristics of the material being inflated can determine the volume (size) of the protruding accessory once inflated. In another embodiment, penile blood vessels shapes are formed upon inflating the device. In another embodiment, a spiral shape is formed upon inflating the device. In another embodiment, the inflated shape that forms an accessory is made of a material that can be inflated with a pump of the invention. In another embodiment, the inflated shape that forms an accessory is made of a material that resists friction.

In another embodiment, the device comprises a penis shaped material having variable thicknesses that define the rigidity in different areas having different thicknesses. In another embodiment, the thickness of the material that is designed to be inflated is determined according to the material used. In another embodiment, the thickness of the material in areas not intended to bet inflated is determined according to the material used. In another embodiment, the thickness of the material in areas that are partially inflated is determined according to the material used. In another embodiment, the thickness of the material in different areas according to a desired functionality (i.e. the ability to inflate) is determined by one of skill in the art. In another embodiment, the device comprising a penis shaped material comprises areas that become more rigid upon inflation. In another embodiment, the device comprising a penis shaped material comprises areas that increase in volume upon inflation. In another embodiment, the shape, color, texture, and size of the areas in the device that can be inflated vary. In another embodiment, penile blood vessels shapes are formed upon inflating the device. In another embodiment, a spiral shape is formed upon inflating the device. In another embodiment, the inflated shape that forms an accessory is made of a material that can be inflated with a pump of the invention. In another embodiment, the inflated shape that forms an accessory is made of a material that resists friction.

In another embodiment, the pump of the invention can inflate and deflate an accessory of the invention. In another embodiment, the pump of the invention can inflate and deflate an accessory of the invention at a predefined pace. In another embodiment, the pump of the invention can inflate and deflate an accessory of the invention at a predefined speed.

In another embodiment, the pump of the invention is an integral part of the device. In another embodiment, the pump of the invention is made of the same materials as the device. In another embodiment, the pump is made of silicon. In another embodiment, the inflating part of the pump is made of silicon. In another embodiment, the inflating part of the pump comprise testicle or testicles shape. In another embodiment, the inflating part of the pump comprise a ball shape.

In another embodiment, the volume of air or liquid pumped into an accessory of the invention is predefined. In another, embodiment, the pump increases the accessory's volume to its maximum. In another, embodiment, the pump does not increase the accessory's volume to its maximum.

In another embodiment, liquid is pumped. In another embodiment, oil based liquid is pumped. In another embodiment, a viscous liquid is pumped. In another embodiment, a water based liquid is pumped. In another embodiment, the liquid is non-toxic. In another embodiment, viscous liquid increases the rigidity of the device of the invention. In another embodiment, one skilled in the art can prepare the various liquids of the invention. In another embodiment, the liquid is releases through the tip of the device. In another embodiment, the liquid released from the tip of the device resembles male ejaculation. In another embodiment, air is pumped.

In another embodiment, an electrical pump is used to activate an accessory of the invention. In another embodiment, the pump is a rotodynamic pump. In another embodiment, the pump is a positive displacement pump. In another embodiment, the pump is a compressed air-powered diaphragm pump. In another embodiment, the pump is a compressed air-powered double-diaphragm pump. In another embodiment, the pump is a kinetic pump. In another embodiment, the pump is a peristaltic pump. In another embodiment, the pump is a pneumatic pump. In another embodiment, the pump is hand operated. In another embodiment, the pump comprises a manual inflator. In another embodiment, the manual inflator comprises a shape of a testicle. In another embodiment, the pump comprises a manual deflator. In another embodiment, the pump is powered by alternating current. In another embodiment, the pump is powered by direct current. In another embodiment, an electrical system suitable to the device of the invention is well known to one of skill in the art.

In another embodiment, the tube is made of non-toxic materials. In another embodiment, the tube is made of materials comprising plastic. In another embodiment, the tube is made of materials comprising MTSA PVC. In another embodiment, a tube comprising MTSA PVC has a density of 1.1-1.3 kg/m³. In another embodiment, the tube is made of materials comprising Styrene-ethylene/butylene-styrene copolymer (SEBS). In another embodiment, a tube comprising SEBS has a density of 0.8-1 kg/m³. In another embodiment, the tube is made of materials comprising Poly(ethylene vinyl acetate) (EVA). In another embodiment, a tube comprising EVA has a density of 0.8-1 g/cm³. In another embodiment, the tube is made of materials comprising Polyethylene (LDPE). In another embodiment, a tube comprising LDPE has a density of 0.8-1 g/cm³. In another embodiment, the tube is made of materials comprising Ethylene/Polypropylene Statistical Copolymer. In another embodiment, a tube comprising Ethylene/Polypropylene Statistical Copolymer has a density of 0.8-1 g/cm³. In another embodiment, the tube is made of materials comprising Polyurethane thermoplastics (PUT). In another embodiment, a tube comprising PUT has a density of 0.9-1.6 g/cm³.

In another embodiment, the tube is a multi-lumen tube. In another embodiment, the tube is a multi-layered tube. In another embodiment, the tube is a bulb tube. In another embodiment, a bulb tube comprises variable cross-sections (the diameter expands over a restricted length). In another embodiment, the tube is a star-shaped tube. In another embodiment, a star-shaped tube comprises extruded sections that can be of almost any shape.

In another embodiment, the tube comprises an intermediate portion located in between the proximal and the distal ends of the tube. In another embodiment, the tube comprises an elastic deformable body arranged in a hollow in the tube wall. In another embodiment, the tube further comprises sealing elements concurring with the tube. In another embodiment, the tube is suitable to produce a flow passage for a fluid, such as a liquid. In another embodiment, the tube is provides suitable means of flow passage for a fluid, such as a liquid. In another embodiment, the tube is suitable to produce a flow passage for air. In another embodiment, the tube is provides suitable means of flow passage for air.

In another embodiment, the invention provides that the device is used for sexual relaxation. In another embodiment, the invention provides that the device allows the user to achieve orgasm faster. In another embodiment, the invention provides that the device allows the user to achieve orgasm easier. In another embodiment, the invention provides that the device allows the user to achieve a stronger orgasm than those produced by manual stimulation alone. In another embodiment, the invention provides that the device is used by women. In another embodiment, the invention provides that the device is used by men. In another embodiment, the invention provides that the device is recommended by sex therapists for women. In another embodiment, the invention provides that the device is recommended by sex therapists for men. In another embodiment, the invention provides that the device is recommended by sex therapists for people having difficulty reaching orgasm by other means. In another embodiment, the invention provides that the device is used by couples as an enhancement to the pleasure of one or both partners. In another embodiment, the invention provides that the device is used by one subject on another subject. In another embodiment, the invention provides that the device is operated by one subject to sexually please another subject.

In another embodiment, the device is made of a single material. In another embodiment, the device is made of a multiple materials. In another embodiment, the device comprises latex. In another embodiment, the device comprises nitrile rubber. In another embodiment, the device comprises rubber with latex. In another embodiment, the device comprises vinyl. In another embodiment, the device comprises cloth. In another embodiment, the device comprises leather. In another embodiment, the device comprises rubber. In another embodiment, the device comprises neoprene. In another embodiment, the device comprises metal. In another embodiment, the device comprises kevlar. In another embodiment, the device comprises materials that exhibit toughness and environmental protection with a degree of sensitivity and flexibility. In another embodiment, the device comprises materials that are non-toxic. In another embodiment, the device comprises materials that are non-carcinogenic.

In another embodiment, device as used herein comprise all the components and elements of the device. In some embodiments, electrical parts are made of metals known to one of skill in the art. In another embodiment, the device comprises a Cyberskin. In another embodiment, Cyberskin mimics the feel of skin. In another embodiment, the device comprises UR3. In another embodiment, the device comprises NewSkin. In another embodiment, the device comprises Ultraskin. In another embodiment, the device comprises jelly. In another embodiment, the device comprises silicone. In another embodiment, the device comprises thermoplastic Rubber (TPR). In another embodiment, the device comprises neoprene. In another embodiment, the device nitrile. In another embodiment, the device comprises polyurethane. In another embodiment, the device comprises a thermal plastic elastomer. In another embodiment the device comprises a mixture of PVC and silicone. In another embodiment, the device comprises an elastomer. In another embodiment, the device comprises a mixture of PVC and rubber. In another embodiment, the device comprises vinyl. In another embodiment, the device comprises polyvinyl chloride and silicone.

In another embodiment, the device comprises a tractor-tread grip. In another embodiment, the device comprises elevated abrasion. In another embodiment, the device is made of materials that are flexible and, tear, and puncture resistant. In another embodiment, the device comprises a fabric lining. In another embodiment, the device is alcohol resistant. In another embodiment, the device is oil resistant. In another embodiment, the device is caustic resistant. In another embodiment, the device is salt resistant. In another embodiment, the device is ketone resistant. In another embodiment, the device is detergent resistant. In another embodiment, the device is petroleum-based solvent resistant. In another embodiment, the device is animal fats resistant.

In another embodiment, the present invention provides that the device 10 comprising a body 12, at least one accessory 30 imbedded in that body 12 and at least one pump 20, wherein at least one tube 22 located within body 12 operatively connects at least one accessory 30 to at least one accessory pump 20. In another embodiment, the activation of at least one accessory pump 20 alters at least one aspect of at least one accessory 30. In another embodiment, accessory 30 is inflatable. In another embodiment, accessory 30 is pushed outwards by an inflatable element. In another embodiment, accessory 30 is attached to an inflatable element. In another embodiment, accessory 30 is not inflatable and pushed outwards by an inflatable element (FIG. 1B). In another embodiment, the activation of at least one accessory pump 20 increases the volume of at least one accessory 30.

In another embodiment, representative embodiment of the present invention may be seen in FIGS. 1A and 1B, wherein FIG. 1A shows a device 10 whose accessories 30 are not inflated and FIG. 1B shows the same device 10 with a plurality of inflated accessories 30.

In another embodiment, the terms “accessory” and “accessories” refer herein to one or more objects that may be affixed to or otherwise appended to the body 12 of a device 10 in order to enhance the sensation produced by that device. In another embodiment, the present invention also enables the user to release fluid substances such as, for example, liquids, creams, and gels from device 10.

In another embodiment, the present invention may be implemented by performing or completing manually, automatically, or a combination thereof, selected steps or tasks. In another embodiment, device 10 of the present invention comprises an electric motor that is preferably battery powered, but may also be powered by other sources such as being plugged into an electric wall outlet. In another embodiment, electric motor or plurality of motors supply power to a pump, vibrating element, electroshocking element, illuminating element, heating element, or a combination thereof. In a preferred embodiment, the device of the invention does not include a vibrating means and the pumping mechanism which causes a change in shape in at least one aspect or portion of the device or accessory is sufficient for sexual sensation. In a preferred embodiment, the device of the invention does not include a motor vibrating means and the pumping mechanism which causes a change in shape in at least one aspect or portion of the device or accessory is sufficient for sexual sensation. In a preferred embodiment, the device of the invention does not include an electronic or electric Vibrating means and the pumping mechanism which causes a change in shape in at least one aspect or portion of the device or accessory is sufficient for sexual sensation. In a preferred embodiment, the device of the invention does not include any electric driven component, thus the pumping mechanism which causes a change in shape in at least one aspect or portion of the device or accessory is sufficient for sexual sensation. In a preferred embodiment, the pumping mechanism is a manual pumping mechanism. In a preferred embodiment, the pumping mechanism is a manual pumping mechanism as described in the figures.

In another embodiment, the present invention provides that device 10 produces pressure in a body cavity. In another embodiment, the present invention provides that device 10 produces pressure and vibration in a body cavity. In another embodiment, the present invention provides that dildo vibrating element is known to one of skill in the art. In another embodiment, the present invention provides that device 10 produces pressure and heat in a body cavity. In another embodiment, the present invention provides that device 10 produces vibratory impulses in body 12. In another embodiment, the present invention provides that body 12 comprises a substantially conical configuration. In another embodiment, the present invention provides that body 12 comprises terminating in a substantially rounded distal end.

In another embodiment, the present invention provides that device 10 is manually operated. In another embodiment, the present invention provides that device 10 is inserted into a body cavity. In another embodiment, the present invention provides that once device 10 is inserted into a body cavity, pumping will increase the volume of the accessories thus causing sexual stimulation. In another embodiment, the present invention provides that once device 10 is inserted into a body cavity, pumping will increase the diameter of the device thus causing sexual stimulation. In another embodiment, the present invention provides that once device 10 is inserted into a body cavity, pumping will increase the diameter of an accessory thus causing sexual stimulation. In another embodiment, the present invention provides that sexual stimulation is produced by pressure exerted by the device. In another embodiment, the present invention provides that sexual stimulation is produced by pressure exerted by the device on a location that is stimulated upon pressure. In another embodiment, the present invention provides that sexual stimulation is produced by pressure exerted by an accessory. In another embodiment, the present invention provides that sexual stimulation is produced by pressure exerted by an accessory on a location that is stimulated upon pressure. In another embodiment, the present invention provides that body location that are stimulated upon pressure are known to one of skill in the art.

In another embodiment, the present invention further provides that in the case of an electric device, the device may be activated by a known in the art power switch. In another embodiment, the present invention provides that a power switch may be situated at the base of body 12.

In another embodiment, the present invention provides that accessory 30 is imbedded into the surface of body 12 of device 10. In another embodiment, the present invention provides that accessory 30 may be arranged in a circular pattern on body 12 of device 10. In another embodiment, the present invention provides that accessory 30 may be arranged randomly on body 12 of device 10.

In another embodiment, the present invention provides that the device comprises multiple accessories 30. In another embodiment, the present invention provides that accessories 30 are identical. In another embodiment, the present invention provides that accessories 30 vary in one or more aspects including, inter alia, type, style, shape, size, color, texture, material, and thickness.

In another embodiment, the present invention provides that one or more aspects, such as size and surface texture, of accessories 30 are adjustable according to the preferences of the user. In another embodiment, the present invention provides that such adjustments enable a user of the present invention to choose the dimensions of accessories 30 according to, for example, the degree of friction desired. In another embodiment, the present invention provides that the degree of friction is determined by, for example, the rigidity or surface texture of each accessory 30.

In another embodiment, the present invention provides that accessories 30 are manufactured from the same or different materials in order to create different effects. In another embodiment, the present invention provides that an accessory 30 is of soft material. In another embodiment, the present invention provides that an accessory 30 is of rough material.

In another embodiment the present invention provides that one or more accessories 30 are detachable and re-attachable. In another embodiment, the present invention provides that the positions of two or more accessories 30 may be exchanged.

In another embodiment, the present invention provides that accessories 30 are inflated by at least one pump 20. In another embodiment, the present invention provides that accessories 30 are made of a suitably airtight material. In another embodiment, the present invention provides that some of the materials provided hereinabove are suitable for inflation as will be readily recognized by one of skill in the art. In another embodiment, the present invention provides that accessories 30 are made of certain rubbers. In another embodiment, the present invention provides that accessories 30 are made of silicone. In another embodiment, the present invention provides that accessories 30 are made of a polymer that can be easily inflated. In another embodiment, the present invention provides that accessories 30 are inflated by, for example, pumping air into an inflatable area of each accessory 30. In another embodiment the present invention provides that accessories 30 are inflated by, for example, pumping liquid of the invention into an inflatable area of each accessory 30. In another embodiment, the present invention provides that accessories 30 are made of materials that can withstand the liquid of the invention.

In another embodiment, the present invention provides that device 10 of the present invention is equipped with at least one pump 20. In another embodiment, the present invention provides that device 10 of the present invention is equipped with at least one pneumatic pump 20. In another embodiment, the present invention provides that device 10 of the present invention is equipped with at least one pump 20, situated at the base of body 12. In another embodiment, the present invention provides that device 10 of the present invention is equipped with at least one pump 20, situated at the base of body 12 wherein the inflator comprises a testicle shape. In another embodiment, the present invention provides that at least one pump 20, enables the user to inflate at least one accessory 30

In another embodiment, the present invention provides that pump 20 forces a gas into an inflatable area of each accessory 30, and inflating each accessory 30. In another embodiment, the present invention provides that pump 20 forces a gas into an inflatable area of each accessory 30, and inflating each accessory 30 to a different shape or size or both. In another embodiment, the present invention provides that pump 20 forces a liquid into an inflatable area of each accessory 30, and inflating each accessory 30. In another embodiment, the present invention provides that pump 20 forces a liquid into an inflatable area of each accessory 30, and inflating each accessory 30 to a different shape or size or both.

In another embodiment, the present invention provides that inflation alters other aspects of each accessory 30 such as, inter alia, surface texture. In another embodiment, the present invention provides that pump 20 is pneumatic; other types of known in the art pumps may also be used.

In another embodiment, the present invention provides that all of accessories 30 are inflated and deflated by a single pump 20. In another embodiment, the present invention provides that all of accessories 30 are only inflated but not deflated by a single pump 20. In another embodiment, the present invention provides that a single pump 20 inflates and deflates a group of accessories 30.

In another embodiment, the present invention provides that device 10 comprises additional pumps 20; each pump 20 being connected to a portion of accessories 30. In another embodiment, the present invention provides that the user of the present invention may thereby choose to, for example, inflate those accessories 30 attached to one pump 20 and while other accessories 30, for example remain deflated or unchanged.

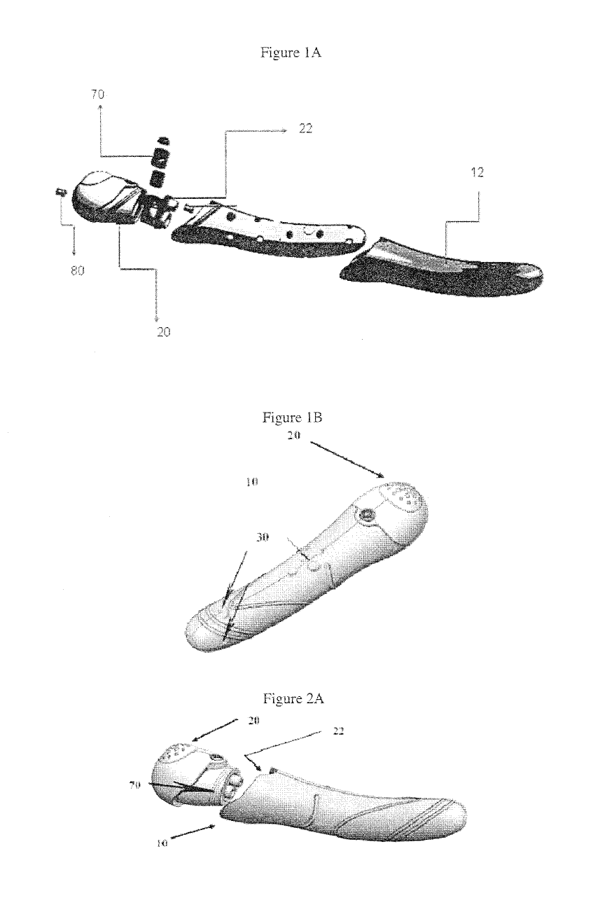

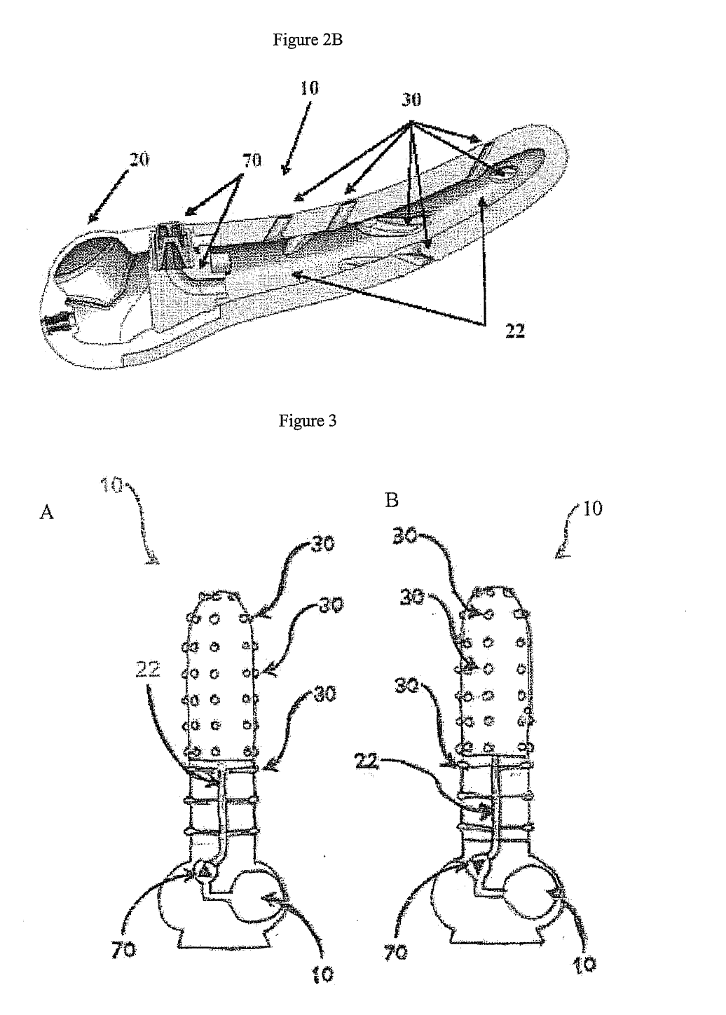

In another embodiment, the present invention provides that a single tube of the invention (such as element 22) operatively connects one or more accessories 30 to a single pump or multiple pumps. Such an embodiment may be seen in FIGS. 2A, 2B, 3A and 3B. In another embodiment, the present invention provides that each inflatable accessory is connected to a pump by a separate tube. In another embodiment, the present invention provides that a first tube operatively connects a first group of accessories to a pump or pumps 20. In another embodiment, the present invention provides that a second tube operatively connects a second group of accessories to a pump or pumps.

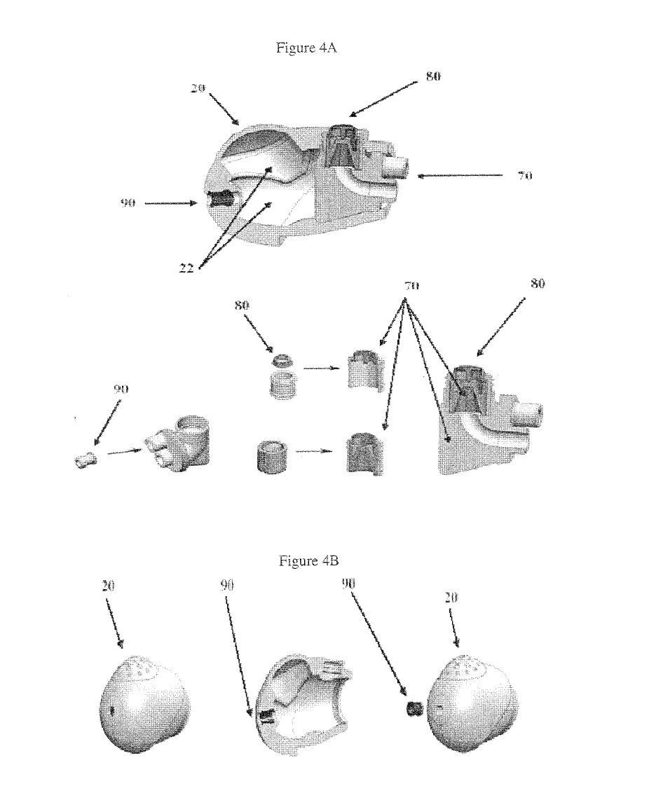

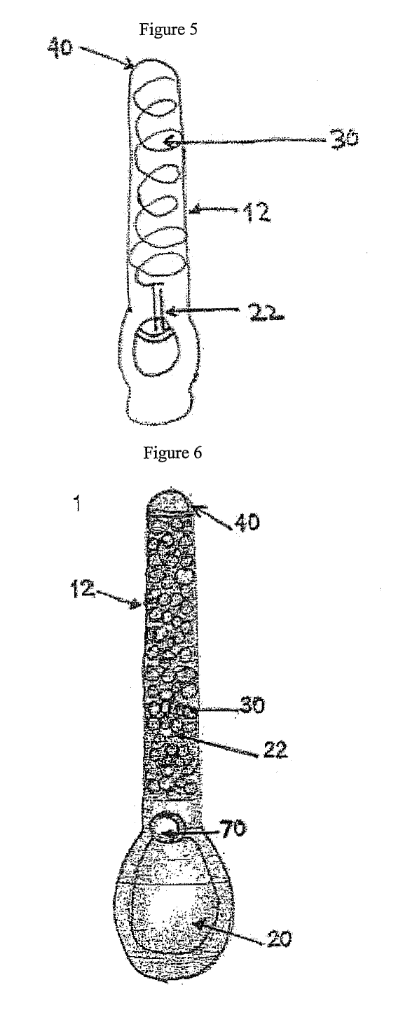

In another embodiment, the present invention provides that device 10 is equipped with more than one accessory pump, in addition to the above connection options, a first tube operatively connects a first group of accessories to a first pump 20, a second tube operatively connects a second group of accessories to a second pump, and so on, wherein each group comprises one or more accessories. Such an embodiment may be seen in FIGS. 4A and 4B.

In another embodiment, the present invention provides that by employing additional tubes that lead from pump to accessories the user is provided with a wide choice of which accessories 30 to alter, and in what combination. In another embodiment, the present invention provides that by employing maneuverable plugs in tubes leading from pump to accessories the user is provided with a wide choice of which accessories to alter, and in what combination.

In another embodiment, the present invention provides that the device is further equipped with a selector switch 70. In another embodiment, the present invention provides that selector switch 70 enables the user to selectively inflate and deflate one or more accessories with a single pump. Such an embodiment is described in FIGS. 3A 3B, 4A, and 4B. In another embodiment, the present invention provides that selector switch 70 enables the user to selectively inflate and deflate one or more accessories with multiple pumps.

In another embodiment, the present invention provides that FIGS. 3A and 4A show the flow of air up into selected accessories 30 through tubes 22. In another embodiment, the present invention provides that FIGS. 3B and 4B show the ebb of the air out of accessories. In another embodiment, the present invention provides that switch system 70 operates manually. In another embodiment, the present invention provides that switch system 70 operates electronically.

In another embodiment, the present invention provides that device 10 comprises a sheath, at least one accessory 30, at least one pump 20, and at least one tube 22 located Within the sheath. In another embodiment, the present invention provides that tube 22 operatively connects accessories 30 to pump 20. In another embodiment, the present invention provides that one or more accessories 30 are imbedded in the sheath and activation of at least one pump 20 alters the shape of at least one accessory 30, as described previously. In another embodiment, the present invention provides that the sheath fits over a conventional device, or a male phallus (not shown).

In another aspect, the present invention provides that some embodiments may be further equipped with a fluid pump, situated towards the base of body 12. In another aspect, the present invention provides that a fluid pump enables the flow of at least one fluid substance, including liquids, creams, gels and the like of various tastes, textures, and smells to an accessory. In another aspect, the present invention provides that a fluid pump enables the release of at least one fluid substance, including liquids, creams, gels and the like of various tastes, textures, and smells outside the device. In another aspect, the present invention provides that a fluid pump enables a pressurized release of at least one fluid substance, including liquids, creams, gels and the like of various tastes, textures, and smells outside the device.

In another embodiment, the present invention provides that a channel operatively connects the fluid pump to a reservoir and to an aperture. In another embodiment, the present invention provides that the channel is located, for example, within body 12. In another embodiment, the present invention provides that the reservoir holds a supply of fluid. In another embodiment, the present invention provides that the aperture provides an exit point for the released fluid. In another embodiment, the present invention provides that the device is a self lubricating device. In another embodiment, the present invention provides that the device is a self lubricating device for use in avoiding irritation or pain during sexual activities or during auto erotic sex activities, while concomitantly increasing the pleasure of the users or user.

In another embodiment, the present invention provides that the liquid is a water-soluble moisturizing fluid, or sterile water or other non-irritating fluid. In another embodiment, the present invention provides that the moisturizing fluid is preferably inert to both plastic and to rubber. In another embodiment, the present invention provides that air or fluid pressure causes the fluid present in a reservoir to exit through the through-hole (apertures) or holes to lubricate any external or internal body parts with which the hole or holes are placed in contact.

In another embodiment, the present invention provides that a fluid reservoir is provided that is either attached directly to the device or remotely connected, say through a tube, but in either case to normally open through-holes to lubricate any external or internal body parts with which the hole or holes are placed in contact with the device. In another embodiment, the present invention provides that the attached or remote reservoir is in fluid contact with the device of the present invention.

In another embodiment, the present invention provides that the diameter of the through-holes added to the device of the present invention for use with lubricating fluids is in the size range of from about 1.5 mm to about 3.5 mm, although smaller or larger diameter holes, say in the range of from about 0.5 mm to about 5 mm may work, depending on the viscosity of the fluid and the energy output of any associated vibrator or other associated stimulation.

In another embodiment, the present invention provides that the device is may be active or passive, say vibrating or non-vibrating.

In another embodiment, the present invention provides that liquids or gels are pumped from the reservoir through at least one channel located, for example, inside device 10 and may exit the channel through at least one aperture on device 10. In another embodiment, the present invention provides that the fluid pump is operated manually. In another embodiment, the present invention provides that the fluid pump is operated electronically.

In another embodiment, the present invention provides that the device, if phallus shaped, is about 5-12″ long. In another embodiment, the present invention provides that the device, if phallus shaped, is about 6-10″ long. In another embodiment, the present invention provides that the device, if phallus shaped, is about 8″ long. In another embodiment, the present invention provides that the widest diameter of the device after inflation is approximately 0.5-1.75″. In another embodiment, the present invention provides that the widest diameter of the device after inflation is approximately 0.8-1.5″. In another embodiment, the present invention provides that the widest diameter of the device after inflation is approximately 0.9-1.4″. In another embodiment, the present invention provides that the widest diameter of the device after inflation is approximately 1.1-1.3″.

In another embodiment, the device comprises a bisque finish. In another embodiment, the device is ergonomic and comfortable.

In another embodiment, the device is further lubricated. In another embodiment, the device is powdered with cornstarch. In another embodiment, the device is powdered with Lycopodium powder. In another embodiment, the device is powdered with talc. In another embodiment, the device is non-powdered. In another embodiment, the device is further lubricated with oil. In another embodiment, the device is further lubricated with Vaseline. In another embodiment, the device is further lubricated with any non-toxic lubricant known to one of skill in the art.

In another embodiment, the invention provides different designs of the device. In another embodiment, the invention provides a multitude of colors. In another embodiment, the invention provides red, black, blue, white, yellow, pink, or a combination thereof devices. In another embodiment, the invention provides that the device is multi-colored.

In another embodiment, the invention provides that the device comprises a different textures for surface play. In another embodiment, the invention provides that the device comprises different textures at various locations. In another embodiment, the invention provides that the device comprises a nubby texture.

In another embodiment, a single inflation event lasts from few seconds up to several hours. In another embodiment, a single inflation event lasts from about 1 second to one ten second. In another embodiment, a single inflation event lasts from about 10 seconds to 20 seconds. In another embodiment, a single inflation event lasts from about 20 seconds to 40 seconds. In another embodiment, a single inflation event lasts from about 30 seconds to 60 seconds. In another embodiment, a single inflation event lasts from about 50 seconds to 90 seconds. In another embodiment, a single inflation event lasts from about 1 minute to 5 minutes. In another embodiment, a single inflation event lasts from about 5 minutes to 15 minutes. In another embodiment, a single inflation event lasts from about 10 minutes to 30 minutes. In another embodiment, a single inflation event lasts from about 20 minutes to 60 minutes. In another embodiment, a single inflation event lasts from about 30 minutes to 45 minutes.

In another embodiment, the device is used to stimulate a female sex organ. In another embodiment, the device is used to stimulate a male sex organ. In another embodiment, the device is used to stimulate the clitoris. In another embodiment, the device is used to stimulate the labia. In another embodiment, the device is used to stimulate the anal area.

In another embodiment, the device further comprises a vibrating means. In another embodiment, the terms vibrating means, vibrating device or rotating means are used interchangeably. In another embodiment, the device's vibrating means comprises rotating means. In another embodiment, the device comprises a single vibrating means or element.

In another embodiment, the device comprises a vibrator element which is a mechanical element that is designed to generate vibrations. In another embodiment, vibration is generated by an electric motor. In another embodiment, vibration is generated by an electric motor with an unbalanced mass on its driveshaft. In another embodiment, when the motor is switched on, the rotating weight resonates.

In another embodiment, the vibration is sine sweep. In another embodiment, the vibration is random vibration. In another embodiment, the vibration is synthesized shock. In another embodiment, a sine sweep vibration profile typically starts vibrating at low frequency and increases in frequency at a set rate (measured in hertz per minute). In another embodiment, the amplitude (measured in gs) of the sine wave may increase or decrease as well. In another embodiment, a random vibration profile will excite different frequencies along a spectrum at different times.

In another embodiment, the vibration element rotates at 10-30000 RPM. In another embodiment, the vibration element rotates at 10-100 RPM. In another embodiment, the vibration element rotates at 100-150 RPM. In another embodiment, the vibration device rotates at 150-300 RPM. In another embodiment, the vibration element rotates at 300-600 RPM. In another embodiment, the vibration element rotates at 600-1000 RPM. In another embodiment, the vibration element rotates adjustment wheel at 600-1500 RPM. In another embodiment, the vibration element rotates adjustment wheel at 1000-3000 RPM. In another embodiment, the vibration element rotates adjustment wheel at 3000-5000 RPM. In another embodiment, the vibration element rotates adjustment wheel at 5000-7500 RPM. In another embodiment, the vibration element rotates adjustment wheel at 7500-10000 RPM. In another embodiment, the vibration element rotates adjustment wheel at 10000-15000 RPM. In another embodiment, the vibration element rotates adjustment wheel at 15000-25000 RPM. In another embodiment, the vibration element rotates adjustment wheel at 25000-30000 RPM.

In another embodiment, the pump and/or vibration element comprises a starting voltage of 0.2-10Vmax. In another embodiment, the pump and/or vibration element comprises a starting voltage of 0.2-1Vmax. In another embodiment, the pump and/or vibration element comprises a starting voltage of 1-3Vmax. In another embodiment, the pump and/or vibration element comprises a starting voltage of 2-5Vmax. In another embodiment, the pump and/or vibration element comprises a starting voltage of 5-10Vmax.

In another embodiment, the vibration element comprises a rated load current of 0.02-5 A. In another embodiment, the vibration element comprises a rated load current of 0.02-0.1 A. In another embodiment, the vibration element comprises a rated load current of 0.05-1.15 A. In another embodiment, the vibration element comprises a rated load current of 1-3 A. In another embodiment, the vibration element comprises a rated load current of 3-5 A.

In another embodiment, the pump and/or vibration element comprises a starting current of 0.02-30 Amax. In another embodiment, the pump and/or vibration element comprises a starting current of 0.02-0.15 Amax. In another embodiment, the pump and/or vibration element comprises a starting current of 0.1-0.2 Amax. In another embodiment, the pump and/or vibration element comprises a starting current of 0.15-0.3 Amax. In another embodiment, the pump and/or vibration element comprises a starting current of 0.3-0.5 Amax. In another embodiment, the pump and/or vibration element comprises a starting current of 0.5-2 Amax. In another embodiment, the pump and/or vibration element comprises a starting current of 2-5 Amax. In another embodiment, the pump and/or vibration element comprises a starting current of 5-10 Amax. In another embodiment, the pump and/or vibration element comprises a starting current of 10-20 Amax. In another embodiment, the pump and/or vibration element comprises a starting current of 20-30 Amax.

In another embodiment, the pump and/or vibration element are mechanical. In another embodiment, a mechanical device comprises a motor. In another embodiment, a mechanical device comprises at least one motor. In another embodiment, a mechanical device comprises two motors. In another embodiment, the vibration element is an electroshocking element. In another embodiment, electroshocking elements of the invention are known to one of skill in the art. In another embodiment, the pump and/or vibration element are pneumatic devices. In another embodiment, pneumatic devices of the invention are known to one of skill in the art. In another embodiment, the pump and/or vibration element are hydraulic devices. In another embodiment, hydraulic devices of the invention are known to one of skill in the art.

In another embodiment, the pump and/or vibration element comprises a voltage of 0.5-18 Volts. In another embodiment, the pump and/or vibration element comprises a voltage of 0.5-1.5 Volts. In another embodiment, the pump and/or vibration element comprises a voltage of 1.5-3 Volts. In another embodiment, the pump and/or vibration element comprises a voltage of 3-6 Volts. In another embodiment, the pump and/or vibration element comprises a voltage of 5-10 Volts. In another embodiment, the pump and/or vibration element comprises a voltage of 10-15 Volts. In another embodiment, the pump and/or vibration element comprises a voltage of 12-18 Volts.

In another embodiment, a vibration element comprises insulated resistance of 0.5-500 Min MΩ. In another embodiment, a vibration element comprises insulated resistance of 0.5-5 Min MΩ. In another embodiment, a vibration element comprises insulated resistance of 5-20 Min MΩ. In another embodiment, a vibration element comprises insulated resistance of 20-50 Min MΩ. In another embodiment, a vibration element comprises insulated resistance of 50-100 Min MΩ. In another embodiment, a vibration element comprises insulated resistance of 75-125N4 in MΩ. In another embodiment, a vibration element comprises insulated resistance of 125-200 Min MΩ. In another embodiment, a vibration element comprises insulated resistance of 200-300 Min MΩ. In another embodiment, a vibration element comprises insulated resistance of 300-400 Min MΩ. In another embodiment, a vibration element comprises insulated resistance of 400-500 Min MΩ.

In another embodiment, the device further comprises an electroshocking means or element. In another embodiment, the device comprises multiple vibrating means. In another embodiment, the device comprises multiple electroshocking means or elements.

In another embodiment, the device comprises a single vibrating means that causes the entire device to move. In another embodiment, the device comprises a single vibrating means that causes the entire device to vibrate. In another embodiment, the device comprises a single vibrating means that causes the entire device to vibrate periodically. In another embodiment, the pump and the vibrating element are synchronized to operate simultaneously. In another embodiment, a vibrating element used in dildos is known to one of skill in the art.

In another embodiment, the device comprises small electroshocking means or small vibrating means at defined locations on the device. In another embodiment, these small electroshocking means or small vibrating means produce local vibrations or shocks. In another embodiment, local vibrations or shocks are placed on the device to match a female G-spot. In another embodiment, small electroshocking means or small vibrating means are movable throughout the device. In another embodiment, small electroshocking means or small vibrating means can be removed.

In another embodiment, the vibrating means or device gets its buzz from an electrically powered oscillator. In another embodiment, vibrations come from an electric motor attached to a rotating disk, with its weight placed off-center. In another embodiment, the faster the motor turns, the higher the vibrator frequency. In another embodiment, the motor is a solenoid. In another embodiment, solenoid electricity passing through a coil of wire forces a metal slug to vibrate rapidly back and forth. In another embodiment, the speed of vibration is 20-80 hertz oscillations. In another embodiment, the speed of vibration is 30-80 hertz oscillations. In another embodiment, the speed of vibration is 40-70 hertz oscillations. In another embodiment, the speed of vibration is 50-70 hertz oscillations. In another embodiment, the vibrating device can run at a spectrum of speeds. In another embodiment, the vibrating device can run at only one, two or three speed settings.

In another embodiment, the device comprises adjustments to increase the strength of the vibrations and/or inflations. In another embodiment, the device comprises adjustments to increase the strength of the vibrations and/or inflations independently of the speed. In another embodiment, the vibration power is adjusted according to how hard a subject presses the vibrating element against a subject's body.

In another embodiment, the vibrating means comprises resonances. In another embodiment, increasing the speed of a continuously variable model increase the power of the vibrations. In another embodiment, increasing the speed of a continuously variable model decrease the power of the vibrations depending on whether you are approaching or passing the resonance. In another embodiment, the power of the vibrations will slowly increase as a subject turns up the speed, then reach a maximum at the resonance frequency, and slowly fall as the subject continue to turn it up.

In another embodiment, vibrations are controlled with damping. In another embodiment, changing how tightly a subject grips a device changes the amount of damping, allowing a subject to modulate the speed and intensity. In another embodiment, changing how tightly a subject grips a device comprising a vibrating means changes the amount of damping, allowing a subject to modulate the speed and intensity. In another embodiment, inserting or contacting a device or a portion thereof comprising a vibrating device into a vagina or anus changes the speed as the soft tissue touching the vibrator absorbs energy.

In another embodiment, damping is adjustable for comfortable use in the anus or vagina and on clitoris. In another embodiment, a rigid clitoris comprises much less damping than softer and more enveloping anal and vaginal tissue.

In another embodiment, springs are used as an oscillator. In another embodiment, pendulums are used as an oscillator. In another embodiment, a pendulum comprises of a weight at the end of a rod or string. In another embodiment, a pendulum will swing at a rate determined by its length, regardless of the amount of weight on the end. In another embodiment, a long pendulum swings slowly, and a short pendulum swings quickly. In another embodiment, a pendulum's frequency is adjusted by changing its length.

In another embodiment, the vibrator's rotation function is continuous. In another embodiment, the vibrator's rotation function is increased. In another embodiment, the vibrator's rotation function is decreased. In another embodiment, the vibrator's rotation function is in a loop sequence. In another embodiment, the vibration speed ranges from slow to fast. In another embodiment, the vibration speed offers variety of frequencies in between.

In another embodiment, the device comprises a pump control attached to the device. In another embodiment, the device comprises a vibrator control attached to the device. In another embodiment, the device comprises a vibrator and pump control attached to the device. In another embodiment, the control allows users to change the mode of pumping and/or vibration.

In another embodiment, the control allows the device and its elements to remain inflated. In another embodiment, the control allows the device to rotate continuously. In another embodiment, the control allows the pump to increase the frequency of inflations. In another embodiment, the control allows the vibrating means to increase the frequency of vibration. In another embodiment, the control allows the pump to decrease the frequency of inflations. In another embodiment, the control allows the vibrating means to decrease the frequency of vibration. In another embodiment, the control allows different setting for the pump and the vibrating means.

In another embodiment, the control allows different setting for region comprising vibrating means in the device. In another embodiment, the control allows the vibrating means to automatically increase rotation speed and then decrease rotation speed. In another embodiment, the control allows the pump and/or vibrating means to play in a loop of sequence of increasing and decreasing frequency of inflations and/or rotation. In another embodiment, the control allows the vibrating means to increase the frequency of vibration. In another embodiment, the control allows two or more tempos of vibration frequency. In another embodiment, the control allows to set a time for each pumping and/or vibrating mode.

In another embodiment, the vibrating elements are attached to the device. In another embodiment, the vibrating elements are comprised within the device. In another embodiment, the vibrating element is embedded within the device. In another embodiment, the vibrating element is laminated to the device. In another embodiment, the vibrating element is encapsulated within the device.

In another embodiment, the pump is electrically connected to an electrical circuit. In another embodiment, a vibrating element is electrically connected to an electrical circuit. In another embodiment, the vibrating element comprises housing. In another embodiment, the housing is the device's shell.

In another embodiment, the device comprises a base controller that falls into the “standard base” category. In another embodiment, the base controller comprises of a cap at the bottom that can be removed by twisting it in a counterclockwise rotation. In another embodiment, the base controller comprises two parts—one part that twists onto the vibrator shaft, and another part that is used to adjust the vibration levels.

In another embodiment, the device comprises small vibratory elements or motors that are affixed within the device. The vibratory motors are sufficiently small and lightweight that they do not interfere with the movement of the device.

In another embodiment, the device further comprises an electrically powered heater so that heat as well as pumping and vibrations are applied. In another embodiment, the heater is supplied electrical power from a dedicated source of electrical power separated from a power source that energizes the vibratory elements.

In another embodiment, small vibratory elements preferably have a height less than about 0.45 inches. In another embodiment, small vibratory elements preferably have a height less than about 0.35 inches. In another embodiment, the vibratory elements preferably have a height less than about 0.25 inches. In another embodiment, the vibratory elements preferably have a height less than about 0.15 inches. In another embodiment, the vibratory elements preferably have a height less than about 0.10 inches.

In another embodiment, the device comprises a multi-component base. In another embodiment, device comprises a remote battery pack. In another embodiment, the device is connected to a battery pack via an extension or a power cord.

In another embodiment, the control is a dial mounted on the base. In another embodiment, the control is adjusted by turning the dial clockwise to turn up the vibrations, and counterclockwise to turn them down. In another embodiment, the control is a separate controller. In another embodiment, a separate controller is connected to the device with a cord, except in the case of remotely controlled devices.

In another embodiment, a switch or other control for applying electrical power from a power source includes an off position and two on positions. In one of the on positions power is applied via a contact, a switchable conduction path and conductors to apply one level of power to the vibratory means. In another embodiment, another contact on the switch is coupled with at least one circuit element, preferably a series resistance that limits the power applied to the vibratory motors.

In another embodiment, the device can be frequently cleaned. In another embodiment, the device is washed with adult toy cleaner or a mild soap and water solution before each use. In another embodiment, the device is washed with adult toy cleaner or a mild soap and water solution after each use. In another embodiment, the device is washed with adult toy cleaner or a mild soap and water solution before and after each use.

In another embodiment, the device is used unlubricated. In another embodiment, the device is lubricated prior to use. In another embodiment, the lubricant is a water-based lubricant. In another embodiment, the lubricant is a water-based lube. In another embodiment, the lubricant is a silicone lube. In another embodiment, the lubricant is oil. In another embodiment, the lubricant is a petroleum-based lubricant. In another embodiment, the lubricant is water. In another embodiment, the lubricant is a lotion. In another embodiment, the lubricant is a cream. In another embodiment, the lubricant is an ointment. In another embodiment, the lubricant further comprises an anti-infective agent. In another embodiment, the lubricant further comprises an anti-bacterial agent. In another embodiment, the lubricant further comprises an anti-viral agent. In another embodiment, the lubricant is compatible with the material or materials from which the device is comprised from.

In another embodiment, the lubricant is pleasant tasting. In another embodiment, the lubricant is flavored. In another embodiment, the lubricant is long-lasting. In another embodiment, the lubricant is easy to clean. In another embodiment, the lubricant is Astroglide. In another embodiment, the lubricant is the fluid used for inflation by the pump.

In another embodiment, the lubricant is a silicone-based lubricant. In another embodiment, the silicone-based lubricant does not contain any water. In another embodiment, the silicone-based lubricant is completely waterproof. In another embodiment, the silicone lubricants are not used with a device comprised of silicone, Cyberskin, latex, or a combination thereof.

In another embodiment, the lubricant is an oil-based lubricant. In another embodiment, the lubricant comprises vegetable oil. In another embodiment, the lubricant is a petroleum-based lubricant. In another embodiment, the lubricant is made with mineral oil. In another embodiment, the lubricant is made with petroleum jelly. In another embodiment, the lubricant comprises Vaseline. In another embodiment, the lubricant comprises baby oil. In another embodiment, the lubricant comprises K-Y Jelly.

In another embodiment, the present invention further provides a device that covers a phallus. In another embodiment, the present invention further provides a device that covers a phallus like structure. In another embodiment, the present invention provides that phallus like structures are known to one of skill in the art. In another embodiment, the present invention provides that a dildo is a phallus like structure. In another embodiment, the present invention provides that a vibrating dildo is a phallus like structure. In another embodiment, the present invention provides that a phallus is a human phallus.

In another embodiment, the present invention further provides the device is a sheath that covers a phallus or a phallus like structure. In another embodiment, the present invention provides that the device comprises at least one accessory of the invention as described hereinabove. In another embodiment, the present invention provides that the device comprises at least one pump of the invention as described hereinabove. In another embodiment, the present invention provides that the device comprises at least one tube of the invention as described hereinabove. In another embodiment, the present invention provides that the tube is operably connected to the pump. In another embodiment, the present invention provides that the tube is operably connected to an accessory.

In another embodiment, the present invention provides that the tube is located within the sheath. In another embodiment, the present invention provides that the sheath is elastic. In another embodiment, the present invention provides that the accessory is located within the sheath. In another embodiment, the present invention provides that the accessory connected to the sheath. In another embodiment, the present invention provides that the accessory causes enlargement of the phallus or phallus like structure that it covers. In another embodiment, the present invention provides that the sheath is used for sexual stimulation. In another embodiment, the present invention provides that the sheath further comprises a vibrating element. In another embodiment, the present invention provides that the sheath further comprises a heating element.

In another embodiment, the present invention provides that the accessory is imbedded in the sheath. In another embodiment, the present invention provides that activation of the pump alters at least one aspect of the accessory. In another embodiment, the present invention provides that the device comprises multiple accessories. In another embodiment, the present invention provides that altering at least one aspect of an accessory comprises enlarging an accessory.

In another embodiment, the present invention provides that altering at least one aspect of an accessory comprises enlarging an accessory. In another embodiment, the present invention provides that altering at least one aspect of an accessory comprises stiffening an accessory. In another embodiment, the present invention provides that altering at least one aspect of an accessory comprises altering the accessory shape upon inflating it. In another embodiment, the present invention provides that altering at least one aspect of an accessory comprises changing the color of the accessory upon inflating it.

In another embodiment, the present invention provides that altering at least one aspect of an accessory comprises changing the texture of the accessory upon inflating it. In another embodiment, the inflatable accessory shape and/or rigidness and/or texture is adapted to a body cavity. In another embodiment, the inflatable accessory shape and/or rigidness and/or texture is adapted to a spot within the body cavity. In another embodiment, the inflatable accessory shape and/or rigidness and/or texture is adapted to a spot within the body cavity characterized as a sexually stimulating spot. In another embodiment, the inflatable accessory shape and/or rigidness and/or texture is adapted to contact and stimulate a clitoris.

In another embodiment, an inflatable accessory of the invention comprises a predefined balloon shape. In another embodiment, an inflatable accessory of the invention comprises a predefined balloon shape coated with a material of the invention. In another embodiment, the covering material comprises various textures that enhance sexual stimulation. In another embodiment, the covering material comprises various textures that enhance sexual stimulation within a body cavity.

In another embodiment, the accessory once inflated widens at least a segment or an area of the device's diameter by at least 1.1 folds. In another embodiment, the accessory once inflated widens at least a segment or an area of the device's diameter by at least 1.2 folds. In another embodiment, the accessory once inflated widens at least a segment or an area of the device's diameter by at least 1.3 folds. In another embodiment, the accessory once inflated widens at least a segment or an area of the device's diameter by at least 1.4 folds. In another embodiment, the accessory once inflated widens at least a segment or an area of the device's diameter by at least 1.5 folds. In another embodiment, the accessory once inflated widens at least a segment or an area of the device's diameter by at least 1.6 folds. In another embodiment, the accessory once inflated widens at least a segment or an area of the device's diameter by at least 1.7 folds. In another embodiment, the accessory once inflated widens at least a segment or an area of the device's diameter by at least 1.9 folds. In another embodiment, the accessory once inflated widens at least a segment or an area of the device's diameter by at least 2 folds. In another embodiment, the accessory once inflated widens at least a segment or an area of the device's diameter by at least 2.5 folds. In another embodiment, the accessory once inflated widens at least a segment or an area of the device's diameter by at least 3 folds. In another embodiment, the accessory once inflated widens at least a segment or an area of the device's diameter by at least 3.5 folds. In another embodiment, the accessory once inflated widens at least a segment or an area of the device's diameter by at least 4 folds. In another embodiment, the accessory once inflated widens at least a segment or an area of the device's diameter by at least 5 folds. In another embodiment, the accessory once inflated widens at least a segment or an area of the device's diameter by at least 6 folds. In another embodiment, the accessory once inflated widens at least a segment or an area of the device's diameter by at least 7 folds. In another embodiment, the accessory once inflated widens at least a segment or an area of the device's diameter by at least 8 folds.

In another embodiment, the extent of increase of the device's width by inflating at least one accessory is controlled by the user. In another embodiment, the extent of increase of the device's width by inflating at least one accessory is predefined. In another embodiment, the extent of increase of the device's width by inflating at least one accessory is set by the user.

In another embodiment, an inflatable accessory of the invention, once inflated, increases the devices' length. In another embodiment, an inflatable accessory of the invention, once inflated, increases the devices' length by at least 1.05 folds. In another embodiment, an inflatable accessory of the invention, once inflated, increases the devices' length by at least 1.1 folds. In another embodiment, an inflatable accessory of the invention, once inflated, increases the devices' length by at least 1.2 folds. In another embodiment, an inflatable accessory of the invention, once inflated, increases the devices' length by at least 1.3 folds. In another embodiment, an inflatable accessory of the invention, once inflated, increases the devices' length by at least 1.4 folds. In another embodiment, an inflatable accessory of the invention, once inflated, increases the devices' length by at least 1.5 folds. In another embodiment, an inflatable accessory of the invention, once inflated, increases the devices' length by at least 1.6 folds. In another embodiment, an inflatable accessory of the invention, once inflated, increases the devices' length by at least 1.7 folds. In another embodiment, an inflatable accessory of the invention, once inflated, increases the devices' length by at least 1.8 folds. In another embodiment, an inflatable accessory of the invention, once inflated, increases the devices' length by at least 1.9 folds. In another embodiment, an inflatable accessory of the invention, once inflated, increases the devices' length by at least 2 folds. In another embodiment, an inflatable accessory of the invention, once inflated, increases the devices' length by at least 1.1 folds. In another embodiment, an inflatable accessory, of the invention, once inflated, increases the devices' length by at least 2.1 folds. In another embodiment, an inflatable accessory of the invention, once inflated, increases the devices' length by at least 2.5 folds. In another embodiment, an inflatable accessory of the invention, once inflated, increases the devices' length by at least 3 folds.

In another embodiment, the extent of increase of the device's length by inflating at least one accessory is controlled by the user. In another embodiment, the extent of increase of the device's length by inflating at least one accessory is predefined. In another embodiment, the extent of increase of the device's length by inflating at least one accessory is set by the user.

In another embodiment, the accessory once inflated widens at least a segment or an area of the device's diameter by at least 0.1 cm. In another embodiment, the accessory once inflated widens at least a segment or an area of the device's diameter by at least 0.2 cm. In another embodiment, the accessory once inflated widens at least a segment or an area of the device's diameter by at least 0.3 cm. In another embodiment, the accessory once inflated widens at least a segment or an area of the device's diameter by at least 0.4 cm. In another embodiment, the accessory once inflated widens at least a segment or an area of the device's diameter by at least 0.5 cm. In another embodiment, the accessory once inflated widens at least a segment or an area of the device's diameter by at least 0.7 cm. In another embodiment, the accessory once inflated widens at least a segment or an area of the device's diameter by at least 1 cm. In another embodiment, the accessory once inflated widens at least a segment or an area of the device's diameter by at least 1.2 cm.

In another embodiment, the accessory once inflated widens at least a segment or an area of the device's diameter by at least 1.5 cm. In another embodiment, the accessory once inflated widens at least a segment or an area of the device's diameter by at least 1.8 cm. In another embodiment, the accessory once inflated widens at least a segment or an area of the device's diameter by at least 2 cm. In another embodiment, the accessory once inflated widens at least a segment or an area of the device's diameter by at least 3 cm. In another embodiment, the accessory once inflated widens at least a segment or an area of the device's diameter by at least 4 cm. In another embodiment, the accessory once inflated widens at least a segment or an area of the device's diameter by at least 5 cm. In another embodiment, the accessory once inflated widens at least a segment or an area of the device's diameter by at least 6 cm. In another embodiment, the accessory once inflated widens at least a segment or an area of the device's diameter by at least 8 cm. In another embodiment, the accessory once inflated widens at least a segment or an area of the device's diameter by at least 10 cm.

In another embodiment, the accessory once inflated lengthens the device by at least 0.1 cm. In another embodiment, the accessory once inflated lengthens the device by at least 0.2 cm. In another embodiment, the accessory once inflated lengthens the device by at least 0.3 cm. In another embodiment, the accessory once inflated lengthens the device by at least 0.4 cm. In another embodiment, the accessory once inflated lengthens the device by at least 0.5 cm. In another embodiment, the accessory once inflated lengthens the device by at least 0.6 cm. In another embodiment, the accessory once inflated lengthens the device by at least 0.7 cm. In another embodiment, the accessory once inflated lengthens the device by at least 0.8 cm. In another embodiment, the accessory once inflated lengthens the device by at least 0.9 cm. In another embodiment, the accessory once inflated lengthens the device by at least 1 cm. In another embodiment, the accessory once inflated lengthens the device by at least 1.2 cm. In another embodiment, the accessory once inflated lengthens the device by at least 1.5 cm. In another embodiment, the accessory once inflated lengthens the device by at least 1.8 cm.

In another embodiment, the accessory once inflated lengthens the device by at least 2 cm. In another embodiment, the accessory once inflated lengthens the device by at least 2.2 cm. In another embodiment, the accessory once inflated lengthens the device by at least 2.6 cm. In another embodiment, the accessory once inflated lengthens the device by at least 3 cm. In another embodiment, the accessory once inflated lengthens the device by at least 2 cm. In another embodiment, the accessory once inflated lengthens the device by at least 3.5 cm. In another embodiment, the accessory once inflated lengthens the device by at least 4 cm. In another embodiment, the accessory once inflated lengthens the device by at least 4.5 cm. In another embodiment, the accessory once inflated lengthens the device by at least 5 cm. In another embodiment, the accessory once inflated lengthens the device by at least 5.5 cm. In another embodiment, the accessory once inflated lengthens the device by at least 6 cm. In another embodiment, the accessory once inflated lengthens the device by at least 6.5 cm. In another embodiment, the accessory once inflated lengthens the device by at least 7 cm. In another embodiment, the accessory once inflated lengthens the device by at least 7.5 cm. In another embodiment, the accessory once inflated lengthens the device by at least 8 cm. In another embodiment, the accessory once inflated lengthens the device by at least 9 cm. In another embodiment, the accessory once inflated lengthens the device by at least 10 cm. In another embodiment, the accessory once inflated lengthens the device by at least 12 cm. In another embodiment, the accessory once inflated lengthens the device by at least 14 cm. In another embodiment, the accessory once inflated lengthens the device by at least 18 cm. In another embodiment, the accessory once inflated lengthens the device by at least 20 cm.

In another embodiment, the present invention provides a kit comprising a device of the invention. In another embodiment, the present invention provides a kit comprising a device of the invention and batteries. In another embodiment, the present invention provides a kit comprising a device of the invention and a lubricant. In another embodiment, the present invention provides a kit comprising a device of the invention and a cleaning material. In another embodiment, the present invention provides a kit comprising a device of the invention and printed instructions. In another embodiment, the present invention provides a kit comprising a device of the invention and printed safety instructions of the device. In another embodiment, the present invention provides a kit comprising a device of the invention and printed operation manual of the device. In another embodiment, the present invention provides a kit comprising a device of the invention and printed cleaning instructions. In another embodiment, the present invention provides a kit comprising a device of the invention and a warranty.

In another embodiment, the present invention provides that while the invention has been described with respect to a limited number of embodiments, these should not be construed as limitations on the scope of the invention, but rather as exemplifications of some of the embodiments. Those skilled in the art will envision other possible variations, modifications, and applications that are also within the scope of the invention. Accordingly, the scope of the invention should not be limited by what has thus far been described, but by the appended claims and their legal equivalents. Therefore, it is to be understood that alternatives, modifications, and variations of the present invention are to be construed as being within the scope and spirit of the appended claims.

What is claimed:

1. A device having a male sex organ shape for providing sexual stimulation in a subject, comprising:
a. an inflatable accessory;
b. a pump comprising an inflator operably coupled to a tube;
c. a tube having a proximal end and a distal end, wherein said distal end of said tube is operably coupled to said pump, and said proximal end of said tube is operably linked to said accessory, wherein upon actuating said pump the volume of said accessory, is increased.

2. A device comprising:
a. a body;
b. at least one accessory;
c. at least one pump; and
d. at least one tube located within the body and operatively connecting said accessory to said pump;
wherein said accessory is imbedded in the body and the activation of at least one pump alters at least one aspect of said accessory.

3. The device of claim 1 or 2, wherein the pump is a manual pump pneumatic.

4. The device of claim 1 or 2, wherein the pump is a pneumatic pump.

5. The device of claim 1 or 2, wherein said pump is a fluid pump.

6. The device of claim 5, wherein said pump releases at least one fluid substance through a tube located within said body operatively connecting said pump to an aperture in said body.

7. The device of claim 1 or 2, wherein said accessory is constructed from an airtight, inflatable material.

8. The device of claim 1 or 2, wherein said accessory is detachable and re-attachable.

9. The device of claim 1 or 2, wherein said accessory is inflated and deflated by a single pump.

10. The device of claim 1 or 2, wherein said accessory is deflated by a maneuverable plug.

11. The device of claim 1 or 2, further comprising a vibrator element.

12. The device of claim 1 or 2, wherein said alter at least one aspect of said accessory comprises increasing said accessory volume or changing said accessory geometrical shape.

13. A device comprising:
a. a sheath;
b. at least one accessory;
c. at least one pump; and
d. at least one tube located within the sheath and operatively connecting said accessory to said pump;
wherein said accessory is imbedded in the sheath and the activation of at least one pump alters at least one aspect of said accessory.

14. The device of claim 13, wherein said sheath fits over at least a conventional dildo or a male phallus.

15. The device of claim 13, further comprising a fluid pump for releasing least one fluid substance.

16. The device of claim 13, wherein said accessory is detachable and re-attachable.

17. The device of claim 13, wherein said accessory is inflated and deflated by a single pump.

18. A kit comprising the device of claim 1 or 2 and a lubricant.
